(19) United States
(12) Patent Application Publication
(10) Pub. No.: US 2007/0218531 A1
(43) Pub. Date: Sep. 20, 2007

(54) Micro-Organism and a Method of Producing Paclitaxel by the Use of the Same

(75) Inventors: Jiepeng Chen, Shantou (CN); Shijin Deng, Shantou (CN); Lili Duan, Shantou (CN); Ronghua Wang, Shantou (CN); Sulan Zhao, Shantou (CN); Xuelian Qiu, Shantou (CN); Shaoming Wei, Shantou (CN); Peize Chen, Shantou (CN); Shouxian Cheng, Shantou (CN); Zhuojun Zheng, Shantou (CN); Hongmin Chen, Shantou (CN); Jie Li, Shantou (CN)

Correspondence Address: BIRCH STEWART KOLASCH & BIRCH, PO BOX 747, FALLS CHURCH, VA, 22040-0747, US

(73) Assignees: HAIYI BIOTECH PTE LTD, Singapore (SG); STRAND BIOSCIENCE COMPANY LIMITED, Guangdong Province (CN)

(21) Appl. No.: 11/631,295

(22) PCT Filed: Jul. 18, 2005
(86) PCT No.: PCT/SG05/00237

§ 371 Date: Jan. 3, 2007

(30) Foreign Application Priority Data
Jul. 19, 2004 (CN) 200410069347.8

(51) Int. Cl. C12P 17/02; C12N 1/14; C07D 305/12

(52) U.S. Cl. 435/123; 549/510; 435/254.11

(57) ABSTRACT

There is provided a new strain, i.e. the alternaria alternata var. monosporus ST-026R CGMCC No. 0899, and its use in the production of paclitaxel. The invention also provides a method for the production of paclitaxel by using the strain CGMCC No. 0899.

### FIELD OF THE INVENTION

The present invention relates to a new micro-organism and a method of producing paclitaxel or derivative thereof by the use of such micro-organism.

### BACKGROUND OF THE INVENTION

Paclitaxel (the chemical name is 5β, 20-epoxy-1,2α,4,7β 10β 13α-hexahydroxy-tax-11-en-9-one-4,10-diacetate-2-benzoate-13-ester with (2R, 3S)-N-benzoyl-3-phenyl isoserine), is currently one of the most efficient anticancer drugs in the world. FDA has approved the clinical application of this drug in the treatment of ovarian cancer, breast cancer, non-small-cell-lung cancer and AIDS-related Kaposi's sarcoma Its curative effect is remarkable Paclitaxel has a unique pharmacological mechanism (i,e., it boosts the concentration of tubulin in cancer cell division, stabilises tubulin and finally leads to the death of cancer cells). It can be used in the treatment of polycystic kidney diseases (American Cytoclonal Pharmaceutics), restenosis associated with bypass vascular surgery (American Boston Scientific Co,) senile dementia, rheumatoid arthritis (Canadian Angiotech Co.) and malaria Remarkable progress has been achieved in these aspects, which indicate the bright future of paclitaxel.

Currently, it is a common practice both in China and abroad to extract paclitaxel or paclitaxel precursors (after that for semi-synthesis of paclitaxel) from Taxus barks or branches and leaves thereof. There exist more than 11 kinds of Taxus trees in the world and they are distributed extensively in the Frigid Zone, Temperate Zone and subtropical zone in Eurasia and North America. However, their distribution is dispersed and they grow very slowly The contents of paclitaxel in Taxus vary from 69/million to 100/million depending on different types of Taxus and different parts of the trees, and the highest content is found in the bark of the short-leave Taxus. The production of 1 kg paclitaxel usually 3,000-4,000 Taxus trees of about 60 years old to be chopped down. In order to maintain ecologic balance and protect the rare Taxus family, countries around the world list Taxus as rare a plant for protection. Therefore, the development of this product is restrained severely by the poor natural resources and restrictions of environment protection. Therefore, there exists a wide gap between the supply and demand of bulk paclitaxel, which makes the price of paclitaxel extremely high.

Many countries have been searching for new ways to produce paclitaxel. Their research efforts are as follows: (1) Chemical semi-synthesis of the compound (Holton, R. A, Somoza, C., Kim, H-B., Liang, F., et al., J. Am. Chem. Soc., 1994, 116 1597; 1994, 116, 1599; Nicolaou, R. C., Yang, Z., Liu, J. J., et al., Nature, 1994, 367, 630; Masters et al., Angew. Chem Int. Ed, Engl., 1995, 34, 1723; Wender, A., Badham Neil F., et al., J. Am, Chem. Soc., 1997, 119, 2755-2758): America gave up producing paclitaxel from the extraction of Taxus barks and leaves in early 1996 and substituted with a semi-synthesis method. The method is first to extract the paclitaxel intermediate, i.e. 10-deacetyl-baccatin (10-DAB) from Taxus leaves and barks, and then the intermediate goes through 2-4 procedures of chemical reactions to synthesise paclitaxel. Although this method protects the environment, it still requires a large number of farms to plant Taxus, which consumes a great deal of land and manpower. It is therefore not a practical way for long term production of paclitaxel. (2) Total sythesis method (Denis J N, Greene A E, Guenard D, et al., J. Am. Chem. Soc., 1988, 110, 5917-1919; Kingston D G Chaudhary A G, Gunatilaka A A L, et al., Tetrahedron Left., 1994, 35, 4483; Kanazawa A M, Denis J N, Greene A E, J. Chem. Soc., Chem. Commun., 1994, 2591; SISTI, Nicholas, J., WO 96/40667; Zhang Hongjie, CN 1241565A): after more than 20 years of efforts, the total synthesis method achieved success in 1994. However, the whole process of the chemical reactions includes over 20 steps. Its efficiency is very low and its cost is very high, so it has no significance for industrial productions (3) Taxus cell tissue culture (Huazhong University of Science and Technology, CN1096820A, Minoru Seki, Chiharu Ohzora, Mayuko Takede, Biotechnology and Bioengineering₇ Vol.53, 214-219, 1997), The method needs a long culture period and it is very difficult to apply in the industrial production. (4) Microbial fermentation (Andrea Stierle, Gray Strobel, W093/21338; Nippon Steel Corporation, WO95104154; Novopharm, WO 96/32490; Gary Strobel, Xianshu Yang, et al., Microbiology, 1996, 142, 435-440; Jianfeng Wang, Guiling Li, et al., FEMS Microbiology Letters, 2000, 193, 249-253): The method is free from the limitations of poor nature resources and ecological protection, but the current strains described in the above international patent applications have the shortcomings of low expression rate of paclitaxel. Therefore, they are not suitable for massive industrial production

Based on the status of paclitaxel production, paclitaxel supply and the paclitaxel demand, there is a need for an alternative and efficient method for mass production of paclitaxel.

### SUMMARY OF THE INVENTION

The present invention addresses the problems above and provides a new strain and a new method to produce mass bulk paclitaxel.

In particular, the strain is named alternaria alternata var. monosporus ST-026R CGMCC No. 0899.

According to a first aspect, the present invention provides a micro-organism, the alternaria alternata var. monosporus ST-026R CGMCC No. 0899.

The invention also provides a cell culture comprising the micro-organism CGMCC No. 0899.

According to another aspect, the present invention provides a method for the production of paclitaxel or a derivative thereof comprising culturing the micro-organism according to the invention. In particular, the derivative expresses a biological activity similar to or the same as that of paclitaxel.

The method according to the invention may comprise incubating the micro-organism in a medium so as to produce and concentrate paclitaxel in the cells of the micro-organism and/or the medium. The method may further comprise collecting and/or purifying the paclitaxel from the cells and/or medium.

The medium may comprise at least one of the following: glucose, sucrose, maltose, fructose, glycerol, starch, lactose, galactose, a carbon source materials that helps the said micro-organism in growing, powder of peanut, powder of soybean, corn steep solid, yeast powder, peptone, beef extract, yeast extract, ammonium nitrate, ammonium chloride, and a nitrogen source materials that helps the said micro-organism in growing.

The medium may comprise a non-organic and/or an organic salt that helps the micro-organism in growing.

The medium may comprise at least one of the following: phosphate, magnesium salt, ferric salt, and sodium salt.

The medium may comprise trace mineral(s) that help the micro-organism in growing.

The medium may comprise at least one of the following: boric acid, potassium iodide, cobalt dichloride, zinc sulphate, and manganese sulphate.

The medium may comprise at least one of the following: methyl jasmonic acid, arachidonic acid, aminocitrate, ceric ammonium nitrate, potassium permanganate, pyruvic acid, p-coumaric acid, vanadic sulfate, hippuric acid, α-naphthylacetic acid, 6-benzylamine purine, silver nitrate, and cinnamic acid.

The medium may comprise at least one of the following: phenylalanine, benzamide, sodium benzoate, sodium acetate, acetamide, propionamide, and benzoic acid,

The medium may comprise at least one of the following:
i) at least one carbon source material selected from the group consisting of glucose, sucrose, maltose, fructose, glycerol, starch, lactose, and galactose;
ii) at least one nitrogen source material selected from the group consisting of powder of peanut, powder of soybean, corn steep solid, yeast powder, peptone, beef extract, yeast extract, ammonium nitrate, and ammonium chloride, phosphate, magnesium salt, ferric salt, and/or sodium salt;
iii) at least one trace mineral selected from the group consisting of boric acid, potassium iodide, cobalt dichloride₇ zinc sulphate, and manganese sulphate;
iv) at least one inducer selected from the group consisting of methyl jasmonic acid, arachidonic acid, aminocitrate, ceric ammonium nitrate, potassium permanganate, pyruvic acid, p-coumaric acid, vanadic sulphate, hippuric acid, α-naphthylacetic acid, 6-benzylamine purine, silver nitrate, and cinnamic acid;
v) at least one precursor, that helps the said microorganism in growing, selected from the group consisting of phenylalanine, benzamide, sodium benzoate, sodium acetate, acetamide, propionamide, benzoic acid, and acetamide,

In particular, in the method according to the invention, the incubation may be made in aerobic conditions at a temperature from 23° C. to 29° C., the fermentative initial pH value is 5.5-11.0, and the pH value is adjusted to 6.0-7.5 during the mid and late fermentative phases

The method according to the invention may comprise at least one inducer, wherein the inducer(s) is added in inoculation, and the inducer concentration is 0.005-0.1% based on the weight of the medium,

The method according to the invention may comprise at least one precursor having a concentration of 0.005-0.1% based on the weight of the medium, and the precursor may be added when the micro-organism growth is at the logarithmic growth phase in the fermentative process.

According to another aspect, in the method of the invention the micro-organism ferments in 7 L and 50 L fermentation tanks with conventional inoculation method or the method of adding mycelia into the fermentation tank, with the carbon source and/or nitrogen source being added successively in the fermentation process, and the pH value is not adjusted in the early fermentation phase but adjusted to 6.0-7.5 during the mid and late phases.

According to another aspect, the method according to the invention may comprise collecting and/or purifying paclitaxel, wherein the steps of collecting and purifying paclitaxel comprise: (1) separating the mycelia and fermenting supernatant from the fermented broth; (2) extracting the obtained mycelia with a first organic solvent and extracting the obtained supernatant with a second organic solvent, and volatilising the solvent; (3) purifying the obtained extract of step (2) with chromatographic purification and crystallisation to obtain the bulk paclitaxel.

In particular, the pH value of the cultured solution before step (1) may be adjusted to 2.0-9.0, using acid or alkaline solution,

Further, the invention provides a method wherein in the chromatographic purification method of step (3), at least one chromatographic column is used, packed with silica gel or aluminium oxide, and the mobile phase includes methanol/methylene dichloride or ethanol/methylene dichloride or acetone/methylene dichloride.

Further, the invention provides a method wherein the crystallisation method used in step (3) comprises: dissolving the crude paclitaxel to be crystallised in methanol, adding water to obtain paclitaxel crystals, and filtrating the crystals; or dissolving the crude paclitaxel to be crystallised in acetone and adding ethanol to obtain paclitaxel crystals, and filtrating the crystals; repeating the above crystallisation process for at least one time; and drying the crystals to get pure bulk paclitaxel.

### BRIEF DESCRIPTION OF THE FIGURES

The patent or application file contains at least one drawing executed in color. Copies of this patent or patent application publication with color drawing(s) will be provided by the office upon request and payment of the necessary fee.

FIG. 1 and FIG. 2 show the spore of new strain according to the present invention.

FIG. 3 shows the mycelia of the new strain according to the present invention.

FIG. 4 is the TLC chart of the sample extracted from the invention's mycelia with acetone and ethyl acetate.

FIG. 5a is the HPLC spectrum of the sample extracted from the invention's mycelia with acetone and ethyl acetate. FIG. 5b is the HPLC spectrum of paclitaxel standard sample,

FIG. 6a is the UV spectrum of the paclitaxel made by the method of present invention. FIG. 6b is the UV spectrum of paclitaxel standard sample.

FIG. 7a is the IR spectrum of the paclitaxel made by the method of present invention FIG. 7b is the IR spectrum of paclitaxel standard sample.

FIG. 8a is the MS spectrum of the paclitaxel made by the method of present invention. FIG. 8b is the MS spectrum of paclitaxel standard sample.

FIG. 9a is the NMR spectrum of the paclitaxel made by the method of present invention. FIG. 9b is the NMR spectrum of paclitaxel standard sample.

FIG. 10 is the chart of the effects of paclitaxel sample made by the method of present invention and standard paclitaxel sample on the killing percentage against ovarian cells by minimal MTT method by the use of culture medium RPMI-1640 containing 2% calf serum.

### DETAILED DESCRIPTION OF THE INVENTION

The whole content of any bibliographic references mentioned in the present specification is herein incorporated by reference.

According to a first aspect, the present invention provides a micro-organism, the alternaria alternata var. monosporus ST-026R CGMCC No. 0899. The strain alternaria alternata var. monosporus ST-026R was deposited with the China General Microbiological Culture Collection Center (CGMCC) on 4 Mar. 2003.

The invention also provides a cell culture comprising the micro-organism CGMCC No. 0899.

According to another aspect, the present invention provides a method for the production of paclitaxel or a derivative thereof comprising culturing the micro-organism according to the invention. In particular, the paclitaxel derivative is a compound which is modified in at least one chemical element or group compared to paclitaxel. However, the derivative expresses a biological activity similar to or the same as that of paclitaxel. The paclitaxel derivative may be prepared by further modifying the strain according to the invention or modifying at least one step of the process of paclitaxel preparation. Accordingly, any reference to paclitaxel encompasses any possible paclitaxel derivative or similar compound.

The method according to the invention may comprise incubating the micro-organism in a medium so as to produce and concentrate paclitaxel in the cells of the micro-organism and/or the medium. The method may further comprise collecting and/or purifying the paclitaxel from the cells and/or medium

The method according to the invention has of shorter cycle, lower cost, and higher production rate. Further, it allows an efficient mass production of paclitaxel if it is used in industry. Its industrial application can not only protect natural resources, but satisfy the requirement of the clinical drug demand as well.

310 samples from the branches, barks, leaves and root barks of 7 Taxus yunnanensis over 300 years old, growing in the conifer and broad-leaf mixed forests at the altitude of 2,500-3,000 m on Laojunshan Mountain, Lijiang, Yunnan Province, China were collected. The barks were sterilised with 60% alcohol in sterile conditions, and cleaned the barks with sterilised water and the remaining water was sucked. The phloem was then torn into thin layers, and the samples were cut into small pieces, and aligned in the order from the phloem to cuticle, inoculate on water agar medium. The samples were kept cultured at a constant temperature of 25° C. for 7-15 days. Different types of microbes grew out consequently, and the colonies of different shapes and different colouirs were inoculated for fermenting experiments. The strains which could produce more paclitaxel were selected and mutated with ultraviolet radiation. After several generations of mutations, a strain of morphologic variation was obtained, which was taken as the basic strain to undergo mutation so as to get the stable monosporous strain for paclitaxel production. The above monosporous strain was achieved through several generations of culture and became comparatively stable, thus the new strain of the invention was obtained.

According to the international code of botanical nomenclature (St. Louis Code), the strain was named as alternaria alternata var, monosporus, with the code of ST-026R. This species has been deposited with the China General Microbiological Culture Collection Center (CGMCC), and its address is No, 13, Beiyitiao, Zhongguancun, Institute of Microbiology, Chinese Academy of Sciences, Beijing, China. The depositing number is CGMCC No. 0899.

The new strain CGMCC No. 0899 provided by the invention has the following microbiological features:

The length and width of the mycelium is roughly equal to each other, or its length is about twice of its width. The cell narrows at its middle part and expands at both ends. It also narrows at the transverse dissepiment. The whole mycelium looks like the form of a chain Its branches are short with sharp angles. The meristem mycelium is short, and is of duplex dichotomy. Most of the spores are single-celled with the size about 20-25 μm. They are colourless and transparent with no verrucous shape. They are in ellipse shape or stick-like shape. The traces of the spores are clear.

The strain of the invention expands in the culture. Its form is densely covered and presents a circular shape with two layers of obvious circular strias. The edge is tidy, the inner layer is dark brown, and the outer layer is off-white. The mycelium is colourless or olive brown with branches and transverse dissepiment. The mycelium has thick and thin mycelium. The mycelium grows fast with abundant spores, and the mycelia can spread all over the surface of 9 cm medium within 5 days. Mycelium forms up wood-like blocks on the surface of the medium. The younger mycelia often form up single or a series of transparent, long cystiform or bead-like spores that is 2-3 times thicker than matured mycelia. The spores can develop into new mycelia. The top of the mycelium is a tipped, thin and transparent long cell.

The strain of the invention produces 3 kinds of spores, the first type being the conidiospore (aerobic spore). Mycelial branches grow out spore chains (every branch top can grow spore chain). The spore chains usually consists of 4-6 spores, fewer of 8-10 spores. The conidiophore generally has 4 cells, and the cells are cylindrical, transparent, solitary and upstanding. The younger conidiospore is egg-like or ellipse with granular spot veins densely distributed on its surface. It is dark coloured and its dissepiment is not clearly visible. Its size is about 21-25×11-15 μm, and the width of the mycelium is 4 μm, the rostrum is 2.0 μm. Spore chains are formed on top or side of the spore. The matured conidiospore is reversed egg-shaped in light colour and is about 25-15 μm, it has 4-6-8 transversing dissepiments and 1-2 longitudinal dissepiments. It narrows between the cells of the spore and the rostrum is 2.0 μm. The second type is gemma, which is produced in the early phase of mycelial growth and in the liquid medium. It is a special spore that symbolises endogenous fungi. It is long crystiform or ball-shaped and it can produce mycelia from its side. The third type is a newly discovered spore that has never been reported yet. It has two parallel mycelium with a zygomite between them. In the middle of the zygomite, a transparent light brown oval spore is produced. A matured spore has 4-6 transverse dissepiments and 2-3 longitudinal dissepiments. Its growing pattern is the same as the zygogamy of alga but different from the sexual production of Rhizopus.

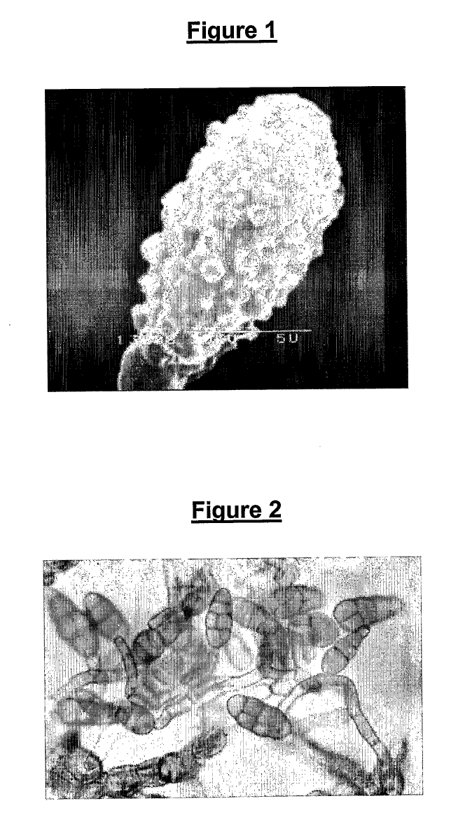

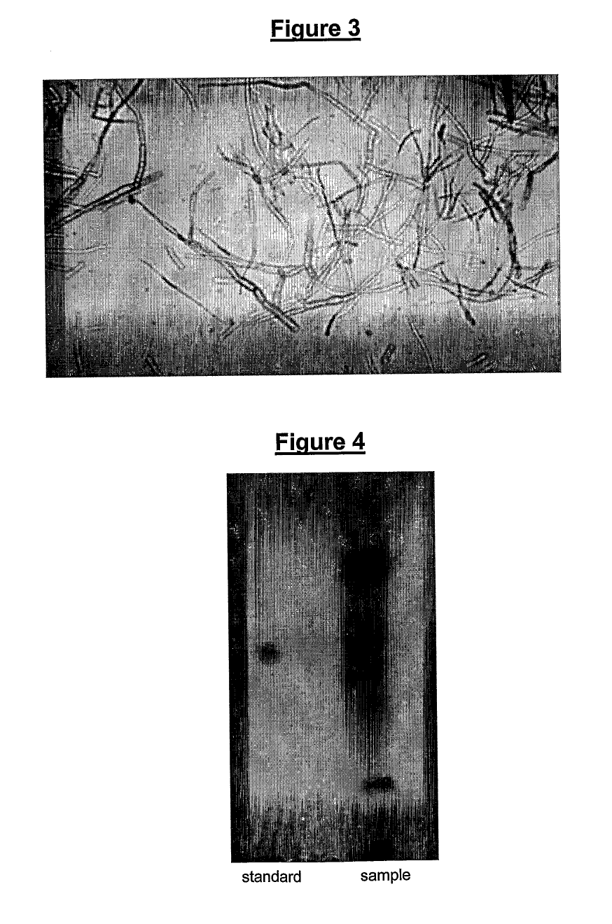

Its shape is as shown in FIGS. 1, 2 and 3.

The invention provides a new method to produce paclitaxel with the new strain CGMCC 0899 of the invention. The method includes: culturing the invention's strain CGMCC 0899 in medium so as to generate and concentrate paclitaxel in the said strain and medium, and collecting and purifying paclitaxel from the said cells and medium.

The said medium can be the conventional or any known media in the field. The said medium comprises carbon source and nitrogen source materials. Other organic or non-organic materials can be added into the medium so as to accelerate microbe's growth and increase the rate of paclitaxel production The said carbon sources include, but are not limited to, glucose, sucrose, maltose, fructose, glycerol, starch, lactose, and/or galactose. The nitrogen sources include, but are not limited to, powder of peanut, powder of soybean, corn steep solid, yeast powder, peptone, beef extract, yeast extract, ammonium nitrate, and/or ammonium chloride. The proper ratio of carbon sources to nitrogen sources is 150:1-40:1. The said non-organic materials include, but are not limited to, phosphate, magnesium salts, such as magnesium sulphate; ferric salts, such as ferrous sulphate, ferric chloride; sodium salts, such as sodium potassium tartrate. The said medium includes, but is not limited to, trace minerals, such as boric acid, potassium iodide, cobalt dichloride, zinc sulphate, manganese sulphate; The said medium includes, but is not limited to, inducers, such as methyl jasmonic acid, arachidonic acid, aminocitrate, ceric ammonium nitrate, potassium permanganate, pyruvic acid, p-coumaric acid, vanadic sulfate, hippuric acid, α-naphthylacetic acid, 6-benzylamine purine, silver nitrate, and cinnamic acid; The said medium includes but not limited to precursors, such as phenylalanine, benzamide, sodium benzoate, sodium acetate, acetamide, propionamide, benzoic acid, and acetamide and etc.

The culture can be conducted under conventional and known conditions in the art. The said fermentation can be under aerobic conditions at the temperature of 23-29° C. with the fermenting initial pH value between 5.5-11.0 (preferably 6.0-7.0). The pH value can be adjusted to 6.0-7.5 in the mid and late phases of fermentation The culture time is determined according to culture conditions, for example, the fermentation can last 6-9 days In the said fermentation period, it is preferable to add the inducers such as methyl jasmonic acid, arachidonic acid, aminocitrate, ceric ammonium nitrate and potassium permanganate into inoculation, and their preferred percentage (with respect to the medium) is 0.005-0.1%. Alkaline solution can be used to adjust the pH value during the fermentation process

The said fermentation can be carried out using conventional or known apparatus and conditions known in the art, for example, oscillating the bottle with conventional or under a known speed rate in the art, or be conducted with the conventional fermentation tanks, such as the 7 L and 50 L tanks

When the 7 L and 50 L tanks are used in the said fermenting process, the carbon source is added successively in the fermentation process, and the pH value is not adjusted in the early fermentation phase but adjusted to 6.0-7.5 during the mid and late phases. The method of adding mycelia into the fermentation tank can be adopted High flow of ventilation can be adopted in the period from inoculation to the early platform phase that the micro-organism grows to, and the ventilation volume can be decreased in the late platform phase.

In order to separate paclitaxel from the medium, any conventional or known separating methods in the art may be used. For example, mycelia can be separated from the fermenting solution with centrifugal or filtrating method; Paclitaxel can be extracted from the solution with one or more organic solvents (such as halogenated hydrocarbon, like methylene dichloride and Chloroform). The paclitaxel in the separated mycelia can be extracted with one or more solvents (such as acetone or methanol). The obtained crude paclitaxel can be purified with one or more conventional or known methods in the fields, such as chromatography and crystallisation.

The method to extract and purify paclitaxel from the said cells and culture medium may comprise the following procedures: (1) Separate the fermented mycelia and supernatant from the culture solution of the said process; (2)

Extract the paclitaxel from the fermented mycelia with the first organic solvent and the supernatant with the second organic solvent, and volatise the solvents completely (3) Purify the obtained matters with chromatography and crystallising method to obtain paclitaxel.

After completion of the said fermentation process, the obtained solution's pH value is adjusted to pH 2-9 with thin acid or alkaline solutions at room temperature or at a temperature lower than the room temperature (the latter is preferable), pre-treated at normal conditions or light-shielding conditions (the latter is preferable) before extracting paclitaxel The conventional or known methods in the art can be adopted for separation in the said procedure (1), for example, the fermented broth can be separated with centrifugal or filtering method.

In the said procedure (2), the fermented mycelia can be dried, mulled and extracted with organic solvent The said drying method can be conventional or known, for example, natural drying, thermal drying under 60° C., vacuum freeze drying, and freeze drying is preferable Light shielding conditions are preferable. The said first organic solvent used for extracting paclitaxel from the fermented mycelia can be any conventional or known solvents, such as acetone, ethyl acetate, methanol, ethanol, methylene dichloride and chloroform, while acetone plus ethyl acetate is preferred The said volatising of the solvent can be conducted with the field's conventional or known methods, such as natural volatising and decompressing concentration.

In the said Procedure (3), the chromatography purification can be conducted with any conventional or known method, the chromatographic column padding can be silica gel or aluminium oxide, and the mobile phase can be methanol/methylene dichloride, ethanol/methylene dichloride or acetone/methylene dichloride. One or more chromatographic columns can be used.

The said crystallisation is conducted by any conventional or known method in the art. The said recrystallisation can be: dissolving the obtained matter into a solvent, such as ethanol, heating it to 40-60° C., adding pure water into it to make paclitaxel crystallise. The temperature is decreased to 4-10° C. and then the obtained matter is filtrated. The above process is repeated till the obtained matter reaches some degrees of purity. It is filtrated and dried in vacuum to get pure paclitaxel. The said recrystallisation can also include dissolving the obtained matter into a solvent, such as acetone, adding a solvent such as hexane to make paclitaxel crystallise. The temperature is decreased to 4-10° C. and it is filtrated. The above process is repeated till the obtained matter reaches some degrees of purity. It is filtrated to get the crystals, the pressure is decreased and the obtained matter is dried to get pure paclitaxel.

Paclitaxel produced by the new strain CGMCC 0899 of the invention mainly exists in the cells (about 70% in the cells and 30% outside of the cells) Therefore, the obtained paclitaxel is of high biomass and high expression rate per liter broth are the goal of the fermenting technology. The method of the invention adopts high nutrition and the most appropriate pH value suitable for growth in the fermenting growth period, and this is the time to accumulate the target material paclitaxel. By adding nutrition and adjusting the pH value, the strain can maintain vigorous life and increase the key enzyme activity for biosynthesis paclitaxel so as to achieve the goal of high expression.

With the new strain CGMCC 0899 and the fermenting method of the invention, one liter broth can produce 120-227 mg crude product of paclitaxel, and its expression rate is much higher than the currently available cell tissue culture method and microbe fermenting method. The technology is characterised by a short cycle, low cost, and high expression rate, and it can thoroughly resolve the technical question of paclitaxel production if it is applied to industrial production. It can both protect the natural environments and meet clinical demand.

While the invention has been described with particular reference to certain embodiments thereof, it will be understood that various modifications can be made to the above-mentioned embodiments without departing from the spirit and scope of the present invention. The examples and the particular proportions set forth are intended to be illustrative only.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001),

### Example 1

The Separation of Micro-Organisms Producing Paclitaxel from Taxus Samples

310 samples from the branches, barks, leaves and root barks of 7 Taxus yunnanensis of over 300 years old at the altitude of 1500-3000 m on Laojunshan Mountain, Lijiang, Yunnan Province were collected The samples were cut into 1 cm×1 cm small pieces, sterilised with 50-90% alcohol for 5 minutes, and homogenised with a homogenizer. The samples were then coated on the PDA culture medium, and were kept at 25° C. for 4-5 days. The mycelia that had grown out were transferred to the slant medium, and cultured at 20-35° C. for 4 days. 1 cm² of mycelia were inoculated in the fermenting culture medium (100 ml for an 250 ml shaking flask, the ingredients of the culture medium: glucose 2%, powder of soybean 0.5%, powder of peanut 0.2%, MgSO₄ 0.01%, KH₂PO₄ 0.05%). The culture was incubated in a rotary shaker at 300 rpm at 25° C. for 16 days. Then it was filtrated with two layers of gauze to get wet mycelia, the wet myceliaare dried in the oven (60° C.) and immersed in chloroform (100 ml chloroform for 20 grams of dry mycelia) for 12 hours. It was then filtered and refrigerated in vacuum to get extractive sample, and 1 ml methanol solution was added. 5 μl of sample was carried on silica gel plate, and extended in three different chromatographic systems (ethyl acetate:isopropyl alcohol=95:5 v/v, chloroform:methanol=7:3 v/v, chloroform:acetonitrile=7:3 v/v), then dried at 60° C. for 30 minutes, and detected with vanillin (vanillin: sulfuric acid: formaldehyde=1:3:96, v/v/v) (100° C., 10 minutes). The mycelia were selected for whose Rf value was the same as the standard sample (Taxol) for further identification. Several microbes separated from Taxus were obtained Several endogenous fungi producing paclitaxel were selected and identified

### Example 2

Screening and Identification of the New Strain

The paclitaxel-producing strain obtained in Example 1 was isolated for three generations (20-35° C., 4-10 days) on PDA culture medium to obtain the ST-026 strain. ST-026 strain grew fast on PDA culture medium and produced abundant spores that are usually black olive green. Conidiospore presented directly from agar culture medium, and presented conidiophore chain from the branches of mycelia. The spore chain consisted of 50-70 spores, the early spores were generated directly from the conidiophore or from 1-2 spore soil surface The spore was stick-like, shaped in an egg, about 22-31 (25)×9-13 (11) μm, with 4-5 transverse dissepiments, 2-3 upright dissepiments or inclined dissepiments.

The initially formed young spores were narrow egg-shaped with densely tiny verrucous protrusions, and because of the dense verrucous protrusions, the transverse dissepiments could not be seen clearly under 100× microscope. The mycelium was colourless or olive green with flat and straight dissepiments, The length of the cell was generally about 5 times the width, and mycelial branches were usually horizontal branched with thin, long and sharp top. According to the above characters and as compared to model species Alternaria alternata founded by Keissler in 1912, the strain was identified as alternaria alternata var. monosporus.

The strain was the basic strain for UV mutation, and after 12 generations of mutations, a morphologically mutant strain UV-012 was obtained, which was taken as the basis for further mutation, and a monosporous strain that produced paclitaxel stably was obtained, and it was coded as ST-026, The above morphological mutant strain was stable after 5 generations of culture. According to the international code of botanical nomenclature, the strain is named as alternaria alternata var. monosporus, and its code is ST-026-R. The stain has been deposited in China General Microbiological Culture Collection Center(CGMCC) and its address is No. 13, Beiyitiao, Zhongguancun, institute of Microbiology, Chinese Academy of Sciences, Beijing, China. The depositing number is CGMCC No. 0899.

### Example 3

Seed Culture

CGMCC No. 0899 lyophilised strain was inoculated in a 250 ml shaking flask containing 100 ml seed culture medium (see Table 1). It was cultured in a rotary shaker at 100-300 rpm for 24-28 hours at 20-30° C. to get the strain ready for inoculation.

TABLE 1
Seed Culture Medium

| Ingredients | % |
|---|---|
| Glucose | 2 |
| Powder of soybean | 0.5 |
| Powder of peanut | 0.2 |
| MgSO4 | 0.01 |
| NaH2PO4 | 0.05 |
| distilled water | 100 ml |
| Adjusted pH value | 7.0-7.2 |

### Example 4

Shaking Flask Fermentation

The seeds obtained in Example 3 were inoculated in production culture medium in an amount of 5-15% based on the weight of the production culture medium (see Table 2), and were cultured for 16 days at 100-350 rpm and at 20-39° C.

TABLE 2
Production culture medium formulations

| Ingredients | % |
|---|---|
| Glucose | 1.5 |
| Powder of soybean | 2.0 |
| Powder of peanut | 0.01 |
| MgSO4 | 0.03 |
| NaH2PO4 | 0.02 |
| distilled water | 100 ml |
| Adjusted pH value | 7.8-8.0 |

The obtained fermented broth was transferred to a 200 ml centrifugal tube and centrifuged for 10 minutes (5000 rpm at 25° C.). The fermented mycelia and fermented supernatant was then collected. The fermented mycelia were incubated in an oven to dry it (60° C. for 16 hours), and milled to mycelial powder. 10 ml acetone was added into each gram of dry mycelial powder, the mixture was immersed and shaken for 12 hours (120 rpm, 25° C.) to get acetone extractive solution, dried in vacuum to get fermented extract.

The same volume of chloroform was added to the obtained fermented supernatant, and the mixture was shaken for 2 hours and it was transferred to a centrifugal tube (8000 rpm, 10 minutes). The mixture was allowed to stand for 10 minutes, sucked the chloroform layer, and the chloroform was dried in low pressure for 10 minute to obtain the extract.

The obtained matter was dissolved with mobile phase, and the solution was added into the chromatographic column packing silica gel (200-400 sieve, the column 5×40 cm high). The mobile phase was 1-5% ethanol/methylene dichloride. It was eluted in the gradient manner Linder medium pressure with a flow rate of 20-60 ml/min; the purified matter was collected and concentrated with reduced pressure, and primary paclitaxel of 92% purity was obtained.

The primary paclitaxel product was dissolved in methanol and heated to 50° C., with water slowly added until paclitaxel was crystallised. The temperature of the solution was slowly reduced to 4° C. and the solution was filtered and then dried in lower pressure to obtain paclitaxel of 99% purity

### Example 5

Shaking Flask Fermentation

The strain obtained in Example 2 was cultured in PDA slant medium, and was directly inoculated with the strain that had been incubated 7 days earlier on the slant medium (containing sucrose 50 g/L, powder of soybean 2.5 g/L, yeast powder 0.5 g/L, magnesium sulphate 0.3 g/L, monopotassium phosphate 0.35 g/L, dipotassium hydrogen phosphate 0.56 g/L, VB1 10 mg/L, ferric trichloride 2 mg/L, manganese sulphate 5 mg/L, zinc sulphate 2.5 mg/L, potassium iodide 0.7 mg/L, boric acid 1.4 mg/L₁ pH 6,8) with the inoculating amount of 2×10⁶ spores/100 ml of production medium Inducers were added at inoculation, and these materials included: 0.01% of arachidonic acid 0.01% based on the weight of the production medium, 0.02% of ceric ammonium nitrate, 0.02% of potassium permanganate.

The amount of production medium was 100 ml/250 ml in shaking flask, culture temperature was 25° C., and rotational speed was 150 rpm. 4 days after inoculation, 0.005% benzamide, 0.01% phenylalanine, 0.04% sodium acetate was added. The fermenting cycle was 9 days. The product was purified by the method of Example 4. Result: the biomass of the fermented broth was 50 g/L, and the target product paclitaxel was 202.3 mg/L.

### Examples 6-9

Shaking Flask Fermentation

The method was the same as Example 5, except that the culture medium's ingredients respectively were:

Example 6. sucrose 50 g/L, powder of soybean 2.5 g/L, yeast extract 0.5 g/L, magnesium sulphate 0.06 g/L, calcium carbonate 1.2 g/L, Vitamin B1 10 mg/L, ferric trichloride 2 mg/L, manganese sulphate 5 mg/L, zinc sulphate 2.5 mg/L, potassium iodide 0.7 mg/L, boric acid 1.4 mg/L, pH 6.8.

Example 7: glucose 10 g/L, sucrose 30 g/L, powder of soybean 10 g/L, yeast extract 0.5 g/L, manganese sulphate 0.1 g/L, Vitamin B1 10 mg/L, dipotassium hydrogen phosphate 0.56 g/L, monopotassium phosphate 0.35 g/L, ferric trichloride 2 mg/L, manganese sulphate 5 mg/L, zinc sulphate 2.5 mg/L, potassium iodide 0.7mg/L, boric acid 1.4 mg/L, pH 6.8.

Example 8: sucrose 40 g/L, powder of soybean 5 g/L, yeast extract 0.5 g/L, magnesium sulphate 1 g/L, dipotassium hydrogen phosphate 0.6 g/L, potassium sodium tartrate 5 g/L, Vitamin B1 10mg/L, ferric trichloride 2 mg/L, manganese sulphate 5 mg/L, zinc sulphate 2.5 mg/L, potassium iodide 0.7 mg/L, boric acid 1.4 mg/L, pH 6.8.

Example 9; glucose 5 g/L, sucrose 20 g/L, dissolved starch 10 g/L, soybean 3 g/L yeast extract 0.5 g/L, magnesium sulphate 0.1 g/L, Vitamin B1 10 mg/L, dipotassium hydrogen phosphate 0.56 g/L, monopotassium phosphate 0.35 g/L, ferric trichloride 2 mg/L, magnesium sulphate 5 mg/L, zinc sulphate 2.5 mg/L, potassium iodide 0.7 mg/L, boric acid 1.4 mg/L, pH 6.8.

The amount of inducers added at the time of inoculation, were respectively as follows:

Example 6: arachidonic acid 0.005%, ceric ammonium nitrate 0.06%, potassium permanganate 0.01%.

Example 7: methyl jasmone 0.01%, ceric ammonium nitrate 0.006, potassium permanganate 0.02%.

Example 8: arachidonic acid 0.05%, ammonium citrate 0.005%, potassium permanganate 0.008%.

Example 9: ammonium citrate 0.05%, ceric ammonium nitrate 0.02%.

The precursors that were added 4 days after inoculation were as follows:

Example 6: benzamide 0.008%, phenylalanine 0.02%, sodium acetate 0.02%.

Example 7; phenylaianine 0.007%, sodium benzoate 0.05%, acetamide 0.01%.

Example 8: benzamide 0.02%, sodium acetate 0.03%, acetamide 0.005%.

Example 9; phenylalanine 0.002%, sodium acetate 0.01%, acetamide 0.09%.

### Example 10

Fermentation in 7 L Tank

The method was the same as that of Example 3, except the culturing temperature was 25° C. and the rotary shaker was at 150 rpm for 24 hours to obtain primary seed mycelia. The method used was that of putting primary seed mycelia directly into the tank for inoculation and the amount of inoculation was 10%, thereby the mycelia were inoculated into the culture medium (glucose 10 g/L, sucrose 20 g/L, powder of soybean 2.5 g/L, yeast powder 0.5 g/L, magnesium sulphate 0.3 g/L, monopotassium phosphate 0.35 g/L, dipotassium hydrogen phosphate 0.56 g/L, VB1 10 mg/L, ferric trichloride 2 mg/L, magnesium sulphate 5 mg/L, zinc sulphate 2.5 mg/L, potassium iodide 0.7 mg/L, boric acid 1.4 mg/L, pH 6.8) in the 7 L tank. Inducers were added at the time of inoculation: 0.01% of arachidonic acid based on the weight of the culture medium, 0.02% of ceric ammonium nitrate, 0.02% of potassium permanganate.

In the early phases of fermentation, the pH value was maintained in its natural stage. 24 hours after the mycelia grew to the platform phase, its pH value was adjusted to 6.0-7.0 with sodium hydroxide solution. This phase needed high flow of ventilation after inoculation and prior to the platform phase (for example, the value of dissolved oxygen was controlled to above 30%), and the air flow was reduced in late platform phase (for example, the value of dissolved oxygen was controlled to lower than 20%). This improved the biosynthesis of paclitaxel. 1% of sucrose was added 36 hours after inoculation, and the addition was for 24 hours continuously. On the 6th day after inoculation, 2% sucrose and 2% maltose were added. When it was fermented for 96 hours, the precursor materials were added in a batch: 0.005% of benzamide, 0.01 of phenylalanine, 0.04% of sodium acetate. The fermenting cycle was 9 days. Result: the production of paclitaxel was 185 mg/L,

### Example 11

Fermentation in 7 L Tank

The strains were obtained from Example 2, and conventional spore slant medium (rice, glucose and agar) was used The slant medium was cultured at 23-29° C. for 5-9 days, and the inoculating amount was 2×10⁷ spores/L medium The spores were inoculated into the 7 L fermenting tank (the medium ingredients comprised the following: glucose 10 g/L, sucrose 20 g/L, powder of soybean 2.5 g/L, yeast extract 0.5 g/L, magnesium sulphate 0.3 g/L, dipotassium hydrogen phosphate 0.35 g/L, monopotassium phosphate 0.56 g/L, VB1 10 mg/L, ferric trichloride 2 mg/L, magnesium sulphate 5 mg/L, zinc sulphate 2.5 mg/L, potassium iodide 0.7 mg/L, boric acid 1.4 mg/L, pH 6.8). 0.01% acetamide was added at the time of inoculation. The pH value was adjusted to 6.0-7.0 in the whole fermenting process with 2N sodium hydroxide and 2N hydrochloric acid. This phase needed high flow of ventilating after inoculation and prior to the platform phase (for example, the value of dissolved oxygen was controlled to over 30%), and the air flow was reduced in late platform phase (for example, the value of dissolved oxygen was controlled to lower than 20%). 1% of sucrose was added 36 hours after inoculation, and the addition was for 24 hours continuously. On the 6th day after inoculation, 1% of culture medium was added and the addition lastED for 24 hours 0.1% acetamide was added during the late platform phase. The fermenting period lasted for 9 days. Result: the production of paclitaxel was 185 mg/L.

### Example 12

Fermentation in 7 L Tank

Fermentation was the same as the method of Example 11, except that the culture medium was: sucrose 50 g/L, powder of soybean 2.5 g/L, yeast extract 0.5 g/L, magnesium sulphate 0.06 g/L, calcium carbonate 3.0 g/L, VB1 10 mg/L, ferric trichloride 2 mg/L, magnesium sulphate 5 mg/L, zinc sulphate 2.5 mg/L, potassium iodide 0.7 mg/L, boric acid 1.4 mg/L, pH 6.8. The inducers added at inoculation were: 0.09% of acetamide based on the weight of the culture medium, 0.01% of potassium permanganate, 0.01% of ceric ammonium nitrate, and 0.005% of arachidonic acid. During the fermenting process, the pH value was controlled to 6.0-7.0 with 2N sodium hydroxide and 2N hydrochloric acid. The value of dissolved oxygen was kept over 30% after inoculation and prior to the platform phase, and lower than 20% after the late platform phase. 0.1% acetamide was added in the late platform phase. The fermenting cycle was 9 days. Result: the production of paclitaxel was 200 mg/L.

### Example 13

Fermentation in 7 L Tank

The seeds are cultured with the method of Example 3. The seed culture medium comprised the following: glucose 20 g/L, powder of soybean 5 g/L, powder of peanut 2 g/L, magnesium sulphate 0.1 g/L, sodium dihydrogen phosphate 0.5 g/L, pH 6.8. The seeds were cultured at 25° C. and the speed of the rotary shaker was 250 rpm for 16 hours

The mycelia were seeded into 7 L fermentation tank by conventional mycelia inoculating method and the inoculating amount was 15% of the culture medium. The culture medium comprised the following: sucrose 20 g/L, powder of soybean 2.5 g/L, yeast extract 0.5 g/L, magnesium sulphate 0.06 g/L, calcium carbonate 3.0 g/L, VB1 10 mg/L, ferric trichloride 2 mg/L, magnesium sulphate 5 mg/L, zinc sulphate 2.5 mg/L, potassium iodide 0.7 mg/L, boric acid 1.4 mg/L, pH 6.8. The following inducers were added at inoculation: acetamide (0.01% based on the weight of the culture medium), potassium permanganate (0.002%), ceric ammonium nitrate (0.005%), arachidonic acid (0.005%). The pH value was maintained between 6.0-7.0 by using 2N sodium hydroxide and 2N hydrochloric acid. The value of dissolved oxygen was kept over 30% after inoculation and prior to the platform phase, and lower than 20% after the late platform phase, 2% sucrose was added 36 hours after inoculation for 24 hours continuously, and 96 hours after inoculation, 0.1% acetamide was added together with 1% culture medium for 12 hours successively The fermenting cycle was 9 days Result: the production of paclitaxel was 189 mg/L.

### Example 14

Fermentation in 7 L Tank

The seeds was fermented according to the method of Example 131 and the following inducers were added at inoculation: potassium permanganate (0.02%), ceric ammonium nitrate (0.05%), arachidonic acid (0.005%). 36 hours after inoculation, 2% sucrose was added for 24 hours successively, and 96 hours after inoculation, 0.01% phenylalanine, 0.04% sodium benzoate, 0.01% acetamide was added along with 1% culture medium drop-wise, The fermenting cycle was 9 days. Result: the production of paclitaxel is 220 mg/L.

### Example 15

Fermentation in 50 L Tank

The seeds were cultured according to the method of Example 3, and the obtained primary seeds were inoculated in culture medium (glucose 10 g/L, sucrose 20 g/L, powder of soybean 2.5 g/L, yeast powder 0.5 g/L, magnesium sulphate 0.3 g/L, monopotassium phosphate 0.35 g/L, dipotassium hydrogen phosphate 0.56 g/L, VB1 10 mg/L, ferric trichloride 2 mg/L, magnesium sulphate 5 mg/L, zinc sulphate 2.5 mg/L, potassium iodide 0.7 mg/L, boric acid 1.4 mg/L, pH 6.8) The following inducers were added at time of the inoculation: arachidonic acid (0.01%), ceric ammonium nitrate (0.05%), potassium permanganate (0.02%). The pH was adjusted to 8.0 in the early phase of fermentation 36 hours after inoculation, 1% sucrose and 0.2% yeast powder was added for 24 hours successively, 6 days after inoculation, 0.005% of benzamide, 0.01% of phenylalanine, 0.04% of sodium acetate was added in one time. The pH value was kept at 6.0-7.0 with 2N sodium hydroxide solution. The fermenting cycle was 9 days. The value of dissolved oxygen was kept higher, at over 30% after inoculation and prior to the platform phase, and later decreased to lower than 20% after the late platform phase, so as to facilitate the synthesis of paclitaxel. Result: the production of paclitaxel was 168 mg/L.

### Example 16

Fermentation in 50 L Tank

The fermentation in SOL tank was carried out according to the method of Example 15 except the following differences were made:

Culture medium of the seeds was: glucose 20 g/L, powder of soybean 5 g/L, powder of peanut 2 g/L, magnesium sulphate 0.1 g/L, sodium dihydrogen phosphate 0.5 g/L, pH 6.8. The seeds are cultured at 25° C. and the rotary shaker was maintained at 250 rpm for 16 hours.

The mycelia were seeded into 50 L fermentation tank by conventional mycelia transferring method and the inoculating amount was 15%. The fermenting culture medium comprised the following: glucose 10 g/L, sucrose 20 g/L, powder of soybean 2.5 g/L, yeast extract 0.5 g/L, magnesium sulphate 0.06 g/L, calcium carbonate 3.0 g/L, VB1 10 mg/L, ferric trichloride 2 mg/L, manganese sulphate 5 mg/L, zinc sulphate 2.5 mg/L, potassium iodide 0.7 mg/L, boric acid 1.4 mg/L, pH 6.8. The following inducers were added at the time of inoculation: acetamide (0.01%), potassium permanganate (0.02%), ceric ammonium nitrate (0.05%), arachidonic acid (0.005%). The pH value was adjusted to 6.0-7.0 with 2N sodium hydroxide and 2N hydrochloric acid. The value of dissolved oxygen was kept over 30% after inoculation and prior to the platform phase, and lower than 20% after the late platform phase. 2% sucrose and 0.1% yeast extract was added 36 hours after inoculation for 24 hours successively, and 96 hours after inoculation, 0.1% acetamide was added along with 1% culture medium for 12 hours successively. The fermenting cycle was 9 days, Result: the production of paclitaxel was 198 mg/L,

### Example 17

Fermentation in 50 L Tank

The fermentation in 50 L tank was carried out according to the method of Example 15 except for the following differences:

The culture medium of the seeds was as follows: glucose 10 g/L, sucrose 30 g/L, soybean 5 g/L, powder of peanut 2 g/L, magnesium sulphate 0.1 g/L, sodium dihydrogen phosphate 0.5 g/L, pH 6.8. The seeds are cultured at 25° C. and the rotary shaker was maintained at 250 rpm for 16 hours

The mycelia were seeded into 50 L fermentation tank by conventional mycelia transferring method and the inoculating amount was 15%. The fermenting culture medium was: glucose 10 g/L, sucrose 40 g/L, powder of soybean 5 g/L, yeast extract 0.5 g/L, magnesium sulphate 0.06 g/L, calcium carbonate 3.0 g/L, Vitamin B₁ 10 mg/L, ferric trichloride 2 mg/L, manganese sulphate 5 mg/L, zinc sulphate 2.5 mg/L, potassium iodide 0.7 mg/L, boric acid 1.4 mg/L, pH 6.8. The following inducers were added at the time of inoculation: acetamide (0.01%), potassium permanganate (0-02%), ceric ammonium nitrate (0.05%), arachidonic acid (0.005%). The pH value was kept at 6.0-7.0 with 2N sodium hydroxide and 2N hydrochloric acid. The value of dissolved oxygen was kept over 30% after inoculation and prior to the platform phase, and lower than 20% after the late platform phase 96 hours after inoculation, 0.1% acetamide was added along with 1% culture medium for 12 hours successively. The fermenting cycle was 9 days Result: the production of paclitaxel was 215 mg/L.

### Example 18

Fermentation in 50 L Tank

The fermentation in 50 L tank was carried out according to the method of Example 15 except for the following differences:

The culture medium of the seeds comprised the following: glucose 10 g/L, sucrose 30 g/L, soybean 5 g/L, magnesium sulphate 0.1 g/L, sodium dihydrogen phosphate 0.5 g/L, pH 6.8. Alternatively, conventional culture medium may also be used. The seeds were cultured at 25° C. and the rotary shaker was maintained at 250 rpm for 16 hours.

The mycelia were seeded into 50 L fermentation tank by conventional mycelia method and the inoculating amount was 15%. The fermenting culture medium was: sucrose 50 g/L, powder of soybean 2.5 g/L, yeast extract 0.5 g/L, magnesium sulphate 0.06 g/L, calcium carbonate 3.0 g/L, Vitamin B₁ 10 mg/L, ferric trichloride 2 mg/L, manganese sulphate 5 mg/L, zinc sulphate 2.5 mg/L, potassium iodide 0.7 mg/L, boric acid 1.4 mg/L, pH 6.8. The following inducers were added: acetamide (0.03%) at inoculation. The pH value was kept at 6.0-7.0 with 2N sodium hydroxide and 2N hydrochloric acid. The value of dissolved oxygen was kept over 30% after inoculation and prior to the platform phase, and lower than 20% after the late platform phase. 96 hours after inoculation, 0.1% acetamide was added in along with 1% culture medium for 12 hours successively. The fermenting cycle was 9 days. Result: the production of paclitaxel was 223 mg/L.

### Example 19

Fermentation in 50 L Tank

The fermentation in 50 L tank was carried out according to the method of Example 15 except for the following differences:

The culture medium of the seeds comprised the following: glucose 20 g/L, soybean 5 g/L, powder of peanut 2 g/L, magnesium sulphate 0.1 g/L, sodium dihydrogen phosphate 0.5 g/L, pH 6-8, The seeds were cultured at 25° C. and the rotary shaker speed was maintained at 250 rpm for 16 hours.

The mycelia were seeded into SOL fermentation tank by conventional mycelia transferring method and the inoculating amount was 15%. The fermenting culture medium was: glucose 10 g/L, sucrose 20 g/L, powder of soybean 2.5 g/L, yeast extract 0.5 g/L, magnesium sulphate 0.06 g/L, dipotassium hydrogen phosphate 0.56 g/L, monopotassium phosphate 0.35 g/L, Vitamin B₁ 10 mg/L, ferric trichloride 2 mg/L, manganese sulphate 5 mg/L, zinc sulphate 2.5 mg/L, potassium iodide 0.7 mg/L, boric acid 1 4 mg/L, pH 6.8. The pH value was kept at 6.0-7.0 with 2N sodium hydroxide and 2N hydrochloric acid. The value of dissolved oxygen was kept over 30% after inoculation and prior to the platform phase, and lower than 20% after the late platform phase. 2% of sucrose and 0.1% of yeast extract was added 36 hours after inoculation for 24 hours successively. 96 hours after inoculation, 0.01% of phenylaianine, 0.04% of sodium benzoate and 0.05% of sodium acetate were added along with 1% culture medium for 12 hours successively. The fermenting cycle was 9 days. Result: the production of paclitaxel was 227 mg/L.

### Examples 20-24

Separation and Purification of the Product

The fermented solution obtained in Examples 10-14 was filtrated with a plate and frame filter-press, and the filtrated mycelia and fermented supernatant was collected. The collected mycelia were dried according to the methods of natural drying, 60° C. heated drying, vacuum freeze-drying, and 100° C. heated drying to obtain dried mycelia 185, 186, 180, 185 and 178 g/7 L respectively. The dried mycelia were milled, and acetone was added (mycelia: solvent=1:20). Also added was ethyl acetate (1:1 or 2:1), acetone, ethanol, methanol and ethyl acetate, respectively, extracting respectively for 24, 26, 30, 30 and 25 hours.

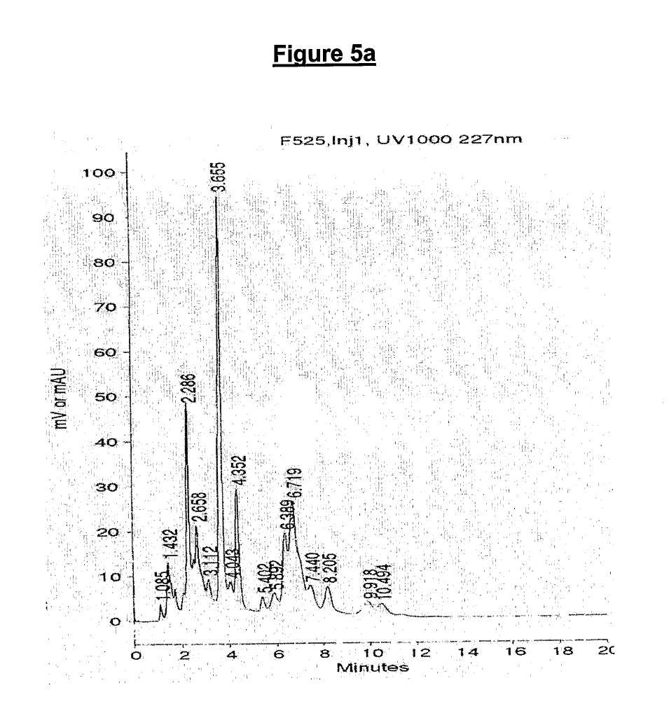

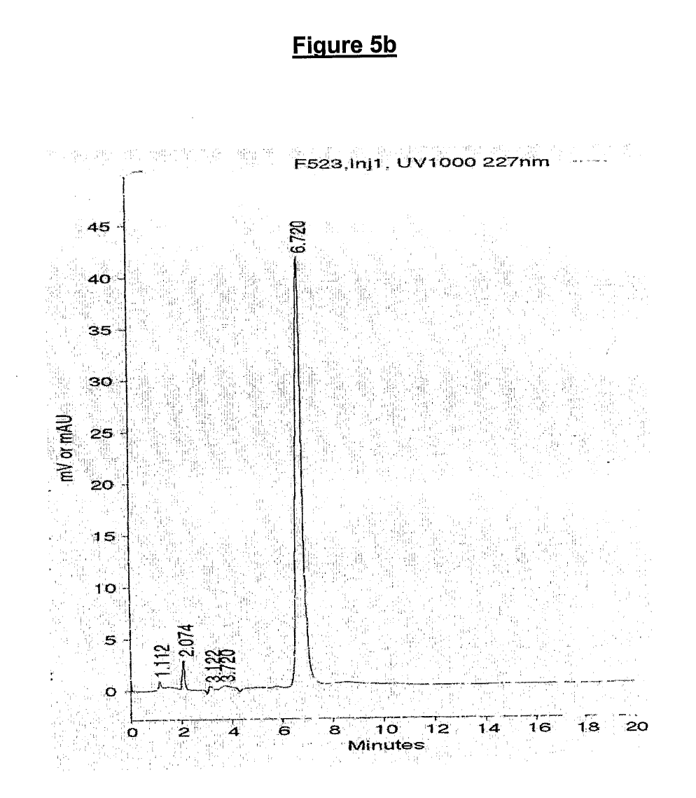

The mixture was the decompressed and the solution was vaporised to obtain the extract. The TLC chart from example 20 is as shown in FIG. 4, and the HPLC chart is as shown in FIG. 5(a). The figures indicate that the samples comprise large contents of paclitaxel.

Same volume of chloroform or methylene dichloride was added into the above obtained supernatant, and the mixtures were extracted for 1-2 hours in vibration, and centrifuged at high speed (8,000 rpm for 10-30 minutes). They were then allowed to stand for 10 minutes, and consequently, the organic solution was separated, decompressed and centrifuged to obtain the extractive matter.

The obtained matter was dissolved with the mobile phase, and the solution was filled into chromatographic columns, packed with: silica gel or aluminium oxide (200-400 sieve, column is 5×40 cm high). The mobile phases were respectively 1%-10% of ethanol/CH₂Cl₂ or methanol/CH₂Cl₂, 1%-30% of acetone/CH₂Cl₂. It was eluted under medium pressure gradient, with a flow speed of 20-60 m/min. The purified products were collected, decompressed and centrifuged to obtain 92% of paclitaxel.

The obtained 92% crude paclitaxel was dissolved in methanol and heated to 40-60° C., by adding water slowly till paclitaxel was crystallised, then decreased the solution's temperature to 4° C.-10° C. and filtrated It was decompressed and dried to get 95% purity of paclitaxel. The above crystallizing process was repeated 2 to 3 times and filtrated, decompressed and dried to obtain 99% purity of paclitaxel.

### Example 25-26

Separation and Purification of the Product

The fermented solutions obtained in Examples 15 and 16 were centrifuged, and the fermented mycelia and the supernatant were collected. The obtained mycelia were lyophilised and milled. Acetone and ethyl acetate (1:1 or 2.1) were added into the dry mycelia powder, dipped and shaken for 12 hours, and filtered to obtain the extracting solution, vaporised rotationally in vacuum to get extractive matter.

Same volume of chloroform or methylene dichloride was added into the supernatant, and the supernatant was extracted for 1-2 hours in vibration, centrifuged in high speed to separate the organic solvent phase, and the organic phase was decompressed and centrifuged to obtain extractive matter,

After dissolving the obtained matter from the previous mycelia or supernatant with mobile phase, the solution was filled into chromatographic columns, packed with silica gel or aluminum oxide (200-400 sieve, and the column was 5×40 cm high). The mobile phase was 1%-10% ethanol/CH₂Cl₂ to be eluted under medium pressure gradient elusion, with a flow speed of 20-60 ml/min. The purified products were collected, decompressed and centrifuged to obtain 92% purity of paclitaxel.

The obtained 92% paclitaxel was dissolved in acetone by slowly adding 1.5-5-time volume of hexane. When paclitaxel began to crystallise, the solution's temperature was slowly reduced to 4° C.-9° C., and filtrated The above crystallizing process was repeated 2 to 4 times and the resultant mixture was filtrated, decompressed and dried to obtain 99% purity of paclitaxel.

### Example 27

The resultant mixture obtained in Examples 17-19 was separated and purified according to the method of Example 25 to get pure paclitaxel products

### Example 28

The resultant mixtures obtained in Examples 12-18 were separated and purified according to the method of Examples 22-27 except for the following treatment: before the fermented solutions obtained in Examples 12-18 were separated, 1 mol/L of HCl or 1 mol/L of NaOH solution was added drop-wise into the fermented solutions and stirred to adjust the pH values to 6.8 (with light shielded in operation), 5.5, 4.5 (with light shielded in operation), 3.0, 7.5 (with light shielded in operation), 8.0 and 6.0 (with light shielded in operation) to obtain pure paclitaxel.

### Example 29

Identification of Paclitaxel Products

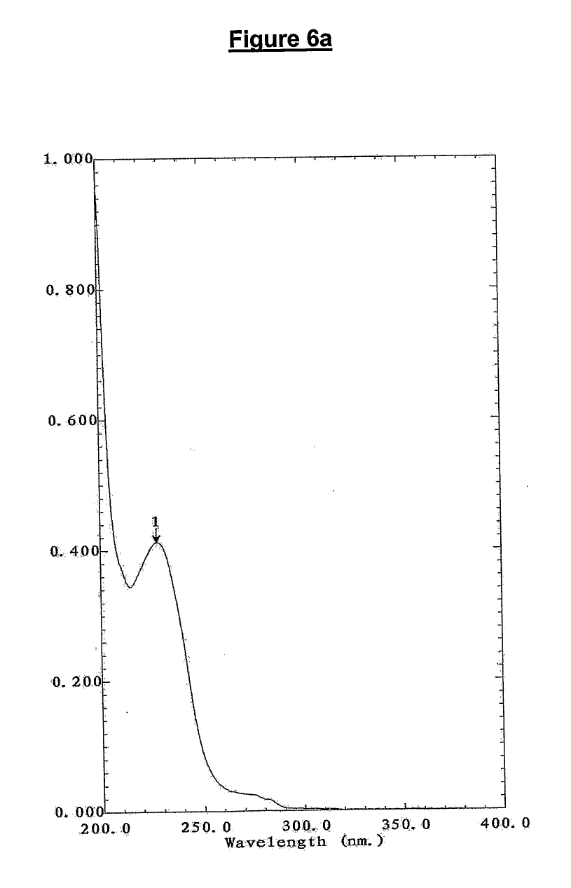

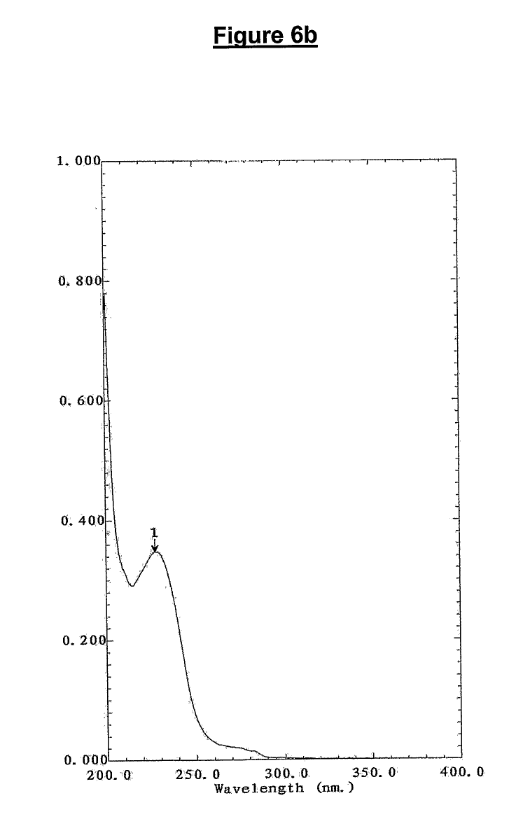

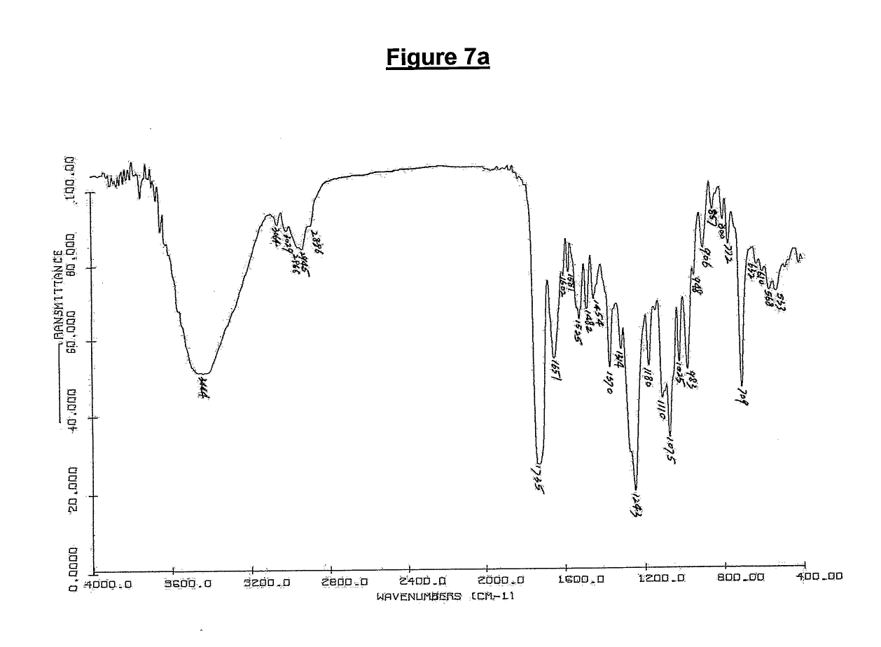

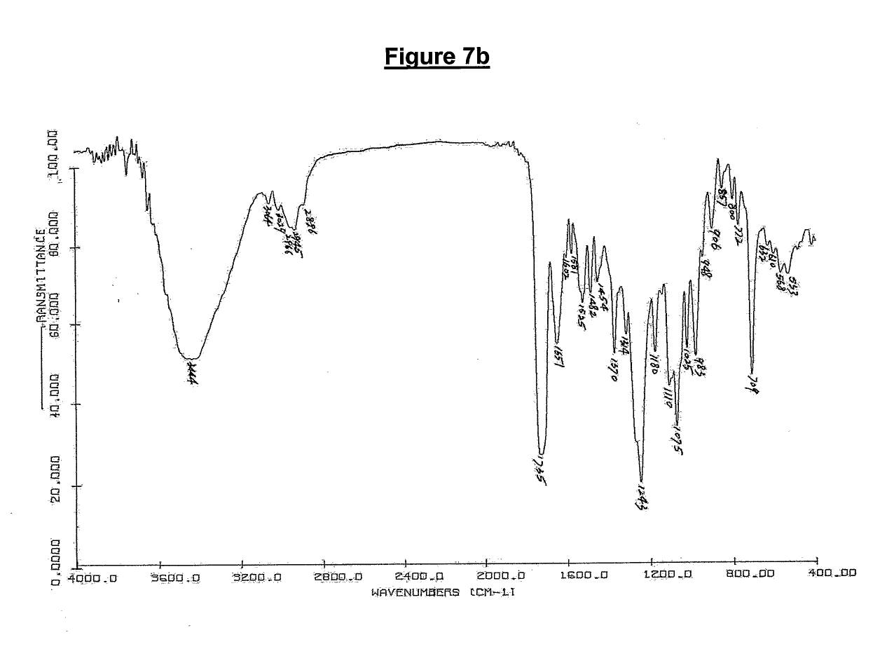

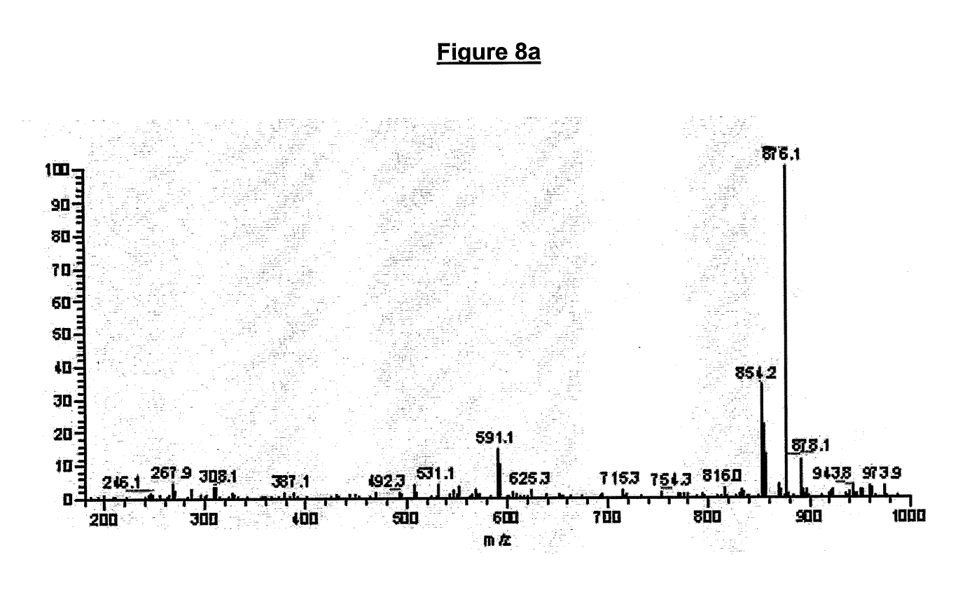

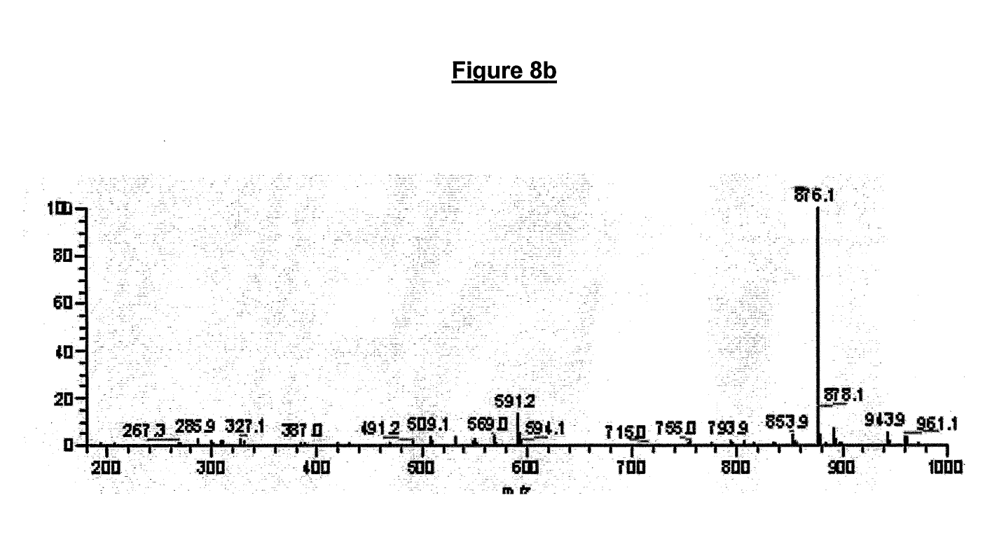

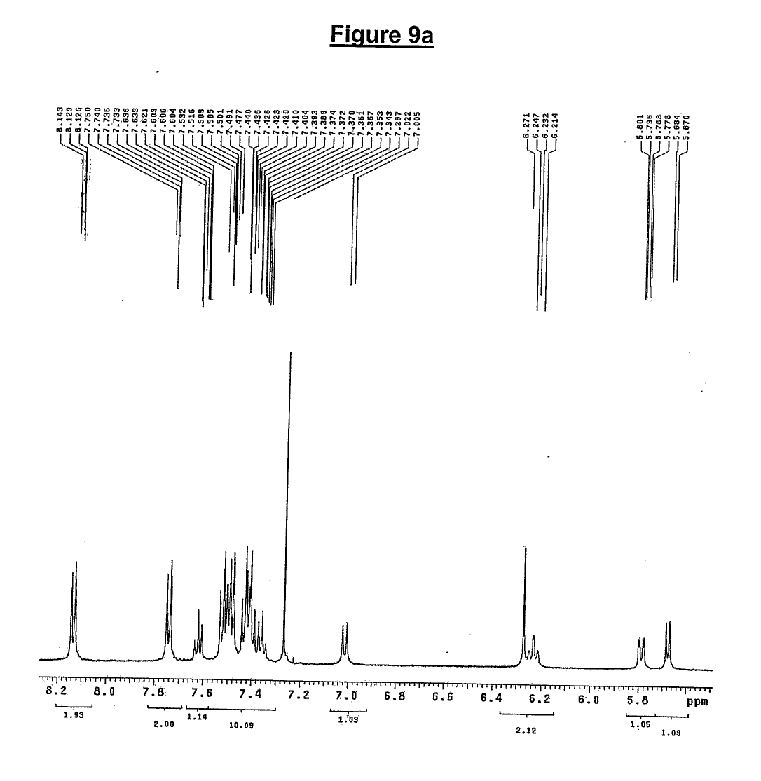

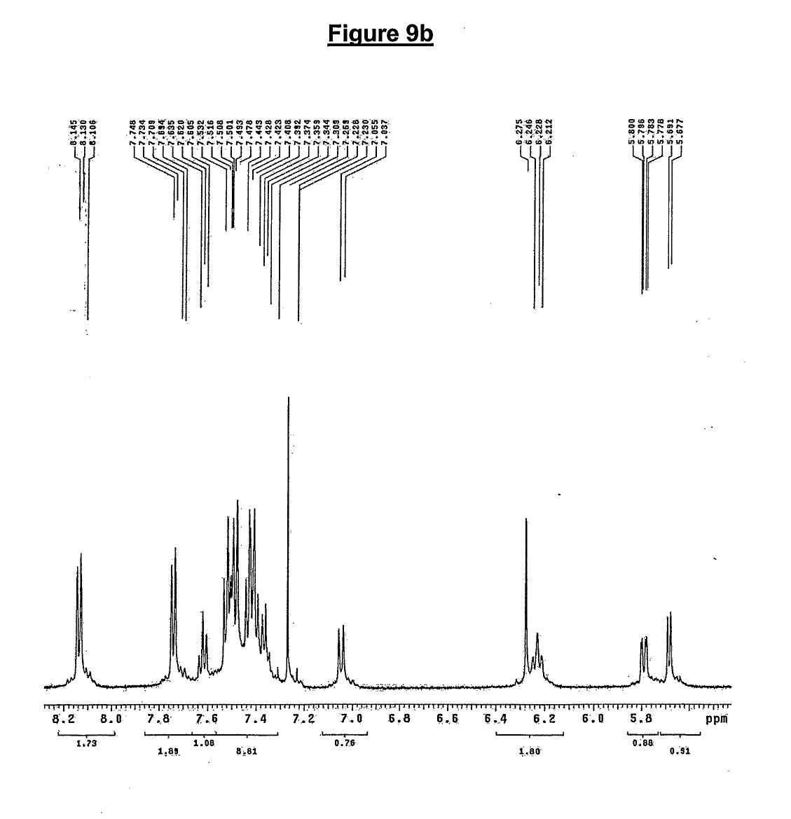

UV, IR, mass spectrum, nuclear magnetic resonance analysis were conducted on the paclitaxel obtained in Examples 20-28, and compared to the standard sample of paclitaxel. The results are shown in FIGS. 6-9. It can be seen from the figures that the fermented paclitaxel according to the method of the present invention are the same as that of paclitaxel standard samples (Paclitaxel T1912 from Sigma Inc.).

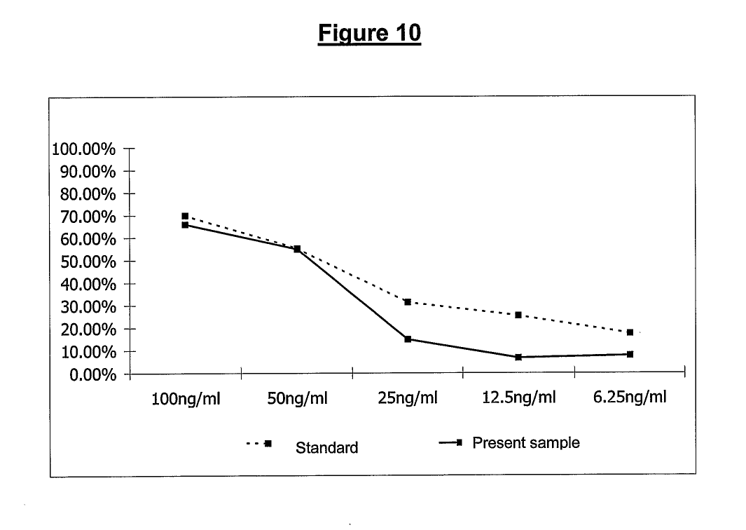

In vitro bioactivity analysis of the fermented paclitaxel show high efficacy on killing breast cancer cells. The results are shown in FIG. 10. The figure shows that the paclitaxel from Examples 20-24 has the same effects as paclitaxel standard samples have.

1-21. (canceled)

22. An isolated micro-organism, the alternaria alternata var. monosporus ST-026R CGMCC No. 0899.

23. A cell culture comprising the micro-organism according to claim 22.

24. A method for the production of paclitaxel or a derivative thereof comprising culturing an isolated micro-organism, wherein the micro-organism is alternaria alternata var. monosporus ST-026R CGMCC No. 0899.

25. The method according to claim 24, comprising incubating the microorganism in a medium so as to produce and concentrate paclitaxel or a derivative thereof in the cells of the micro-organism and/or the medium.

26. The method according to claim 24, further comprising collecting and/or purifying the paclitaxel or a derivative thereof from the cells.

27. The method according to claim 25, further comprising collecting and/or purifying the paclitaxel or a derivative thereof from the medium.

28. The method according to claim 25, wherein the medium comprises at least one of the following: glucose, sucrose, maltose, fructose, glycerol, starch, lactose, galactose, a carbon source material that helps the said micro-organism in growing, powder of peanut, powder of soybean, corn steep solid, yeast powder, peptone, beef extract, yeast extract, ammonium nitrate, ammonium chloride, and a nitrogen source material that helps the said micro-organism in growing.

29. The method according to claim 25, wherein the medium comprises a non-organic and/or an organic salt that helps the micro-organism in growing.

30. The method according to claim 25, wherein the medium comprises at least one of the following: phosphate, magnesium salt, ferric salt, and sodium salt.

31. The method according to claim 25, wherein the medium comprises trace mineral(s) that help the micro-organism in growing.

32. The method according to claim 25, wherein the medium comprises at least one of the following: boric acid, potassium iodide, cobalt dichloride, zinc sulphate and manganese sulphate.

33. The method according to claim 25, wherein the medium comprises at least one of the following: methyl jasmonic acid, arachidonic acid, aminocitrate, ceric ammonium nitrate, potassium permanganate, pyruvic acid, p-coumaric acid, vanadic sulfate, hippuric acid, α-naphthylacetic acid, 6-benzylamine purine, silver nitrate, and cinnamic acid.

34. The method according to claim 25, wherein the medium comprises at least one of the following: phenylalanine, benzamide, sodium benzoate, sodium acetate, acetamide, propionamide, and benzoic acid.

35. The method according to claim 25, wherein the medium comprises at least one of the following:
i) at least one carbon source material selected from the group consisting of glucose, sucrose, maltose, fructose, glycerol, starch, lactose, and galactose;
ii) at least one nitrogen source material selected from the group consisting: of powder of peanut, powder of soybean, corn steep solid, yeast powder, peptone, beef extract, yeast extract, ammonium nitrate, and ammonium chloride, phosphate, magnesium salt, ferric salt, and/or sodium salt;
iii) at least one trace mineral selected from the group consisting of boric acid, potassium iodide, cobalt dichloride, zinc sulphate, and manganese sulphate;
iv) at least one inducer selected from the group consisting of methyl jasmonic acid, arachidonic acid, aminocitrate, ceric ammonium nitrate, potassium permanganate, pyruvic acid, p-coumaric acid, vanadic sulphate, hippuric acid, α-naphthylacetic acid, 6-benzylamine purine, silver nitrate, and cinnamic acid;
v) at least one precursor, that helps the said micro-organism in growing, selected from the group consisting of phenylalanine, benzamide, sodium benzoate, sodium acetate, acetamide, propionamide, benzoic acid, and acetamide.

36. The method according to claim 25, wherein the incubation is carried out under aerobic conditions at a temperature from 23° C. to 29° C., the fermentative initial pH value is 5.5-11.0, and the pH value is adjusted to 6.0-7.5 during the mid and late fermentative phases.

37. The method according to claim 25, wherein at least one inducer is added in inoculation, and the inducer concentration is 0.005-0.1% based on the weight of the medium.

38. The method according to claim 25, wherein at least one precursor having concentration of 0.005-0.1% based on the weight of the medium is added when the micro-organism growth is at the logarithmic growth phase in the fermentative process.

39. The method according to claim 25, wherein the micro-organism ferments in 7 L and 50 L fermentation tanks with conventional inoculation method or the method putting mycelia into the fermentation tank, a carbon source and/or nitrogen source is added successively in the fermentation process, and pH value is not adjusted in the early fermentation phase but adjusted to 6.0-7.5 during the mid and late phases.

40. The method according to claim 26, wherein the steps of collecting and purifying paclitaxel comprise: (1) separating the mycelia and fermenting supernatant from the fermented broth; (2) extracting the obtained mycelia with a first organic solvent and extracting the obtained supernatant with a second organic solvent, and volatilising the solvent; (3) purifying the obtained extract of step (2) with chromatographic purification and crystallisation to obtain the bulk paclitaxel.

41. The method according to claim 40, wherein pH value of the cultured solution before step (1) is adjusted to 2.0-9.0, using acid or alkaline solution.

42. The method according to claim 40, wherein in the chromatographic purification method of step (3), at least one chromatographic column is used, packed with silica gel or aluminium oxide, and the mobile phase includes methanol/methylene dichloride or ethanol/ methylene dichloride or acetone/methylene dichloride.

43. The method according to claim 40, wherein the crystallisation method used in step (3) comprises: dissolving the crude paclitaxel to be crystallised in methanol, adding water to obtain paclitaxel crystals, and filtering the crystals; or dissolving the crude paclitaxel to be crystallised in acetone and adding ethanol to obtain paclitaxel crystals, and filtering the crystals; repeating the above crystallisation process for at least one time; and drying the crystals to get pure bulk paclitaxel.
